SEQUENCE LISTING (6 sequences)

SEQ ID NO: 1 - DNA, 24 bp, Artificial
  Oligonucleotide as a forward primer for amplifying urease B gene using PCR method
atcctagaatgaaaaagattagca

SEQ ID NO: 2 - DNA, 24 bp, Artificial
  Oligonucleotide as a backward primer for amplifying urease B gene using PCR method
gagctcctagaaaatgctaaagag

SEQ ID NO: 3 - DNA, 25 bp, Artificial
  Oligonucleotide as a forward primer for amplifying tissue plasminogen activator using PCR method
aatctagacatggatgcaatgaaga

SEQ ID NO: 4 - DNA, 26 bp, Artificial
  Oligonucleotide as a backward primer for amplifying tissue plasminogen activator using PCR method
atgatctctggtcacggtcgcatgtt

SEQ ID NO: 5 - DNA, 1710 bp, Helicobacter pylori
atgaaaaagattagcagaaaagaatatgtttctatgtatggccctactacaggcgataaagtgagattgggcgatacagacttgatcgctgaagtagaacatgactacaccatttatggcgaagagcttaaattcggcggtggtaaaaccctaagagaaggcatgagccaatctaacaaccctagcaaagaagaactggatctaatcatcactaacgctttaatcgtggattacaccggtatttataaagcggatattggtattaaagatggcaaaatcgctggcattggtaaaggcggtaacaaagacatgcaagatggcgttaaaaacaatcttagcgtgggtcctgctactgaagccttagccggtgaaggtttgatcgtaactgctggtggtattgacacacacatccacttcatctccccccaacaaatccctacagcttttgcaagcggtgtaacaacgatgattggtggcggaactggccctgctgatggcactaacgcaaccactatcactccaggtagaagaaatttaaaatggatgctcagagcggcagaagaatattctatgaacttaagtttcttagctaaaggtaacgcttctaacgatgcaagcttagccgatcaaattgaagccggtgcgattggctttaaaatccacgaagactggggcaccactccttctgcaatcaatcatgcgttagatgttgcggacaaatacgatgtgcaagtcgctatccacacagacactttgaatgaagccggttgtgtagaagacactatggcagccattgccggacgcactatgcacactttccacactgaaggcgctggtggcggacacgctcctgatattattaaagtagctggtgaacacaacattctgcccgcttccactaaccccactatccctttcactgtgaatacagaagcagaacacatggacatgcttatggtgtgccaccacttggataaaagcattaaagaagatgttcagttcgctgattcaaggatccgccctcaaactattgcggctgaagacactttgcatgacatggggattttctcaatcaccagttctgactctcaagctatgggtcgtgtgggtgaagttatcaccagaacttggcaaacagctgacaaaaacaaaaaagaatttggccgcttgaaagaagaaaaaggcgataacgacaacttcaggatcaaacgctacttgtctaaatacaccattaacccagcgatcgctcatgggattagcgagtatgtaggttctgtagaagtgggcaaagtggctgacttggtgttgtggagtcccgcattctttggcgtgaaacccaacatgatcatcaaaggcggattcattgcattgagtcaaatgggtgatgcgaacgcttctatccctaccccacaaccggtttattatagagaaatgttcgctcatcatggtaaagctaaatacgatgcaaacatcacttttgtgtctcaagcggcttatgacaaaggcattaaagaagaattagggcttgaaaggcaagtgttgccggtaaaaaattgcagaaacatcactaaaaaagacatgcaattcaacgacactaccgctcacattgaagtcaatcctgaaacttaccatgtgttcgtggatggcaaagaagtaacttctaaaccagccaataaagtgagcttggcacaactctttagcattttctag

SEQ ID NO: 6 - DNA, 2280 bp, Homo sapiens
ggagtccagggctggagagaaaacctctgcgaggaaagggaaggagcaagccgtgaatttaagggacgctgtgaagcaatcatggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagatgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtgaccaggaacacccgactcctcaaaagcaaatgagatcccgcctcttcttcttcagaagacactgcaaaggcgcagtgcttctctacagacttctccagacccaccacaccgcagaagcgggacgagaccctacaggagagggaagagtgcattttcccagatacttcccattttggaagttttcaggacttggtctgatttcaggatactctgtcagatgggaagacatgaatgcacactagcctctccaggaatgcctcctccctgggcagaaagtggccatgccaccctgttttcagctaaagcccaacctcctgacctgtcaccgtgagcagctttggaaacaggaccacaaaaatgaaagcatgtctcaatagtaaaagataacaagatctttcaggaaagacggattgcattagaaatagacagtatatttatagtcacaagagcccagcagggcctcaaagttggggcaggctggctggcccgtcatgttcctcaaaagcaccct

(19) United States
(12) Patent Application Publication
(10) Pub. No.: US 2005/0150016 A1
(43) Pub. Date: Jul. 7, 2005

(54) Method for producing a recombinant protein using pollen

(76) Inventor: Hee-Sung Park, Gyeongsan-si, Gyeongsangbuk-do (KR)

Correspondence Address: NATH & ASSOCIATES, 1030 15th STREET, NW, 6TH FLOOR, WASHINGTON, DC, 20005, US

(21) Appl. No.: 10/500,447

(22) PCT Filed: Nov. 19, 2002
(86) PCT No.: PCT/KR02/02154

(30) Foreign Application Priority Data
Nov. 19, 2001 (KR) 2001/71712

(51) Int. Cl. A01H 1/00; C12N 15/82

(52) U.S. Cl. 800/294

(57) ABSTRACT

The invention relates to method for producing a recombinant protein using pollen. More specifically, the invention relates to method for producing a recombinant protein by introducing a recombinant gene into pollen using Agrobacterium tumefaciens via vacuum infiltration and then inducing pollen tube growth, and it makes possible to scale-up recombinant protein within short period of time and low production costs.

### TECHNICAL FIELD

The present invention relates to a method for producing recombinant protein using pollen.

More particularly, the present invention relates to a method for producing recombinant protein coding a target gene by introducing the target gene into plant pollen by following transformation.

### BACKGROUND ART

On attempts to produce recombinant protein in transgenic plant systems, much research has been conducted on the production of protein through intracellular accumulation (cytoplasm or intracellular organelle) in specific tissue or organ (seed, tuber, etc.) by employing plant, plant organs, cultured plant cells or secretion systems (intracellular gap, secretion in medium). (Molony, Biotechnol. Eng., 9, 3, 1995; Kusnadi et al., Biotechnol. Bioeng., 56, 473, 1997; Smith & Glick, Biotechnol. Adv., 18, 85, 2000; Sijmons et al., Bio/Technology, 8, 217, 1990; Doran, Curr. Opin. Biotechnol., 11, 199, 2000; Boothe et al., Drug Develop. Res., 42, 171, 1997; Giddings et al., Nature Biotechnol., 18, 1151, 2000).

Recombinant protein synthesizing systems that employ plants provide many advantages over other recombinant protein synthesizing systems employing animal cells or bacteria, such as the moderate cost of cultivation control, feasibility of scale-up, similarity to human glycosylation or folding, absence of product impurities from transgenic animals or bacteria, and absence of contaminations of human-derived proteins.

Recombinant protein production systems that employ transgenic plant cell lines are targeted to produce valuable proteins within a short period in bioreactors. This requires careful control and analysis techniques to operate the bioreactors for the maintenance and proliferation of transgenic plant cell lines.

Regarding an invention that employs plant pollen, U.S. Pat. No. 5,929,300 describes a method for producing transgenic plants by introducing a gene into the plant pollen using Agrobacterium, pollinating with the pistil, obtaining the seeds, and then germinating them to obtain the resulting transformed plant. However, it encountered problems with the long expression period of the target gene from obtaining the seeds from the transformation and inbreeding and because of the complicated process. In addition, only the pollen of annual plants, including tobacco and cotton, could be used, except for the pollen of trees such as fine, gingko tree, etc.

In studying to solve the said problems, the inventor demonstrated that when recombinant gene is introduced into plant pollen via Agrobacterium tumefaciens and vacuum infiltration and then pollen tube growth is induced, it could result in the mass production of recombinant protein within a short period.

Up to now, there are no cases that employ plant pollen as the host for producing recombinant protein. However, the following merits can be expected when pollen is used to produce recombinant protein:

First, a production system with pollen cells can be utilized at a lower price compared to production systems with cell lines because pollen cells can be collected plentifully at lower prices and there are no complicated costs from cell proliferation, maintenance of cellular genetic stability and operation of bioreactors.

Second, the gene can be easily introduced via the method of particle bombardment, vacuum infiltration and electroporation in a production system employing pollen cells (Tjokrokusumo et al., Plant Cell reports, 19, 792, 2000; Fernando et al., Plant Cell reports, 10, 224, 2000).

Third, recombinant protein can be directly produced from pollen growth culture from a few times to a maximum of several days in a simple liquid medium supplemented with 3˜4 kinds of chemicals, including sugar.

Fourth, plant pollen is a very excellent protein-producing host with timely and economical efficiency, since the recombinant protein can be collected in a simple medium according to the use of secretion signals of various kinds of protein secreted by the pollen.

The inventor accomplished the present invention through genetic introduction and expression by recognizing that plant pollen is a producing host of recombinant protein that can produce protein in a short period at lower prices. Therefore, the object of the present invention is to provide a method for producing recombinant protein using plant pollen as a producing host of various kinds of recombinant protein for use in testing, diagnosis and prevention and for industrial use.

### DISCLOSURE OF THE INVENTION

According to the present invention, the object is achieved by collecting plant pollen, culturing them, introducing a target gene into the pollen via vacuum infiltration, and expressing and identifying recombinant protein from the germinated pollen.

The process of the present invention consists of isolating a pollen from the plant, culturing the pollen in a pollen growth medium, inserting a target gene into an Agrobacterium vector and then transforming the Agrobacterium, infecting the transformed Agrobacterium into the cultured plant pollen, and growing the pollen with the target gene to obtain the resulting recombinant protein.

The present invention's method of producing recombinant protein using pollen will be explained in greater detail below:

Step 1: Culturing Plant Pollen

Dehisced anthers were detached from lily flowers (Lilium longiflorum) or pine trees and their pollen grains were collected and then stored in a deep freezer at −70° C. until use. 0.5˜5 g of the pollen grains were suspended in 200 mL of PGM and incubated at 25˜30° C. for 2˜24 hours in the dark.

In the present invention, plant pollen is not limited to specific plant pollens, but all kinds of plant pollen can be used, including Pinaceae, Ginkgoaceae, as well as annual plants such as lily, cotton and tobacco.

In the present invention, the pollen growth medium (PGM) was preferably composed of 5˜10% sucrose, 0.5˜3 mM Ca(NO₃)₂, 50˜300 μM H₃BO₃, 0.001˜5 mM KNO₃ and 0.1˜10 mM KH₂PO₄, more preferably 7% sucrose, 1.27 mM Ca(NO₃)₂, 162 μM H₃BO₃, 0.99 mM KNO₃ and 3 mM KH₂PO₄.

Step 2: Construction of Recombinant Plasmid

A recombinant plasmid was constructed from the target gene by inserting to an Agrobacterium vector.

According to the present invention, ureB gene from Helicobacter pylori and tPA gene from humans were used for the pollen transformation.

In order to produce protein encoded by ureB gene in accordance with the present invention, ureB gene was inserted into pBI121 with GUS reporter gene to obtain pBIUreB, which is the recombinant plasmid for Agrobacterium transformation. To produce protein encoded by tPA (tissue plasminogen activator), the tPA gene was inserted into pBI121 with GUS reporter gene to obtain pBI/tPA or pSK/tPA, which is the recombinant plasmid for Agrobacterium transformation.

The recombinant proteins produced by the method of the present invention comprise some proteins produced from plant pollen, including ureB and tPA proteins.

Step 3: Transformation

The recombinant plasmid prepared in step 2 was introduced to Agrobacterium tumefaciens using the freeze-thaw method, incubated and selected in LB media with antibiotics such as kanamycin or cefotaxime.

Step 4: Introduction of Gene Via Vacuum Infiltration

In accordance with the present invention, the introduction of the gene to the plant pollen using Agrobacterium was carried out via particle bombardment, vacuum infiltration and electroporation.

Step 5: Identifying the Target Gene

Genomic DNA was extracted from germinated pollens with the recombinant gene according to the method of CTAB (Draper J. et al., Plant genetic transformation and gene expression: A laboratory manual, p204-208, Blackwell Scientific Publication, 1988).

The PCR technique can be used to determine whether the target gene was correctly introduced. In addition, the expression of the gene can be confirmed by conventional molecular techniques such as Southern blotting, Northern blotting and Western blotting. Through the molecular techniques, the enzymatic activity of β-glucronidase expressed from pBI121, as well as the expression of the gene, could be assayed by histochemical staining, which turns into a blue color by reacting with a substrate, X-gluc.

In accordance with the present invention, 10 hours to 7 days is required to express recombinant protein by transformation and grow the pollen with the target gene.

The present invention will be explained in greater detail through reference with the following examples. However, the following examples are provided only to illustrate the present invention, and it should be understood that the scope of the present invention is not limited thereto.

### BRIEF DESCRIPTION OF THE INVENTION

The objective, features and other advantages of the present invention will be more clearly understood from the following detailed description taken in conjunction with the accompanying drawings, in which:

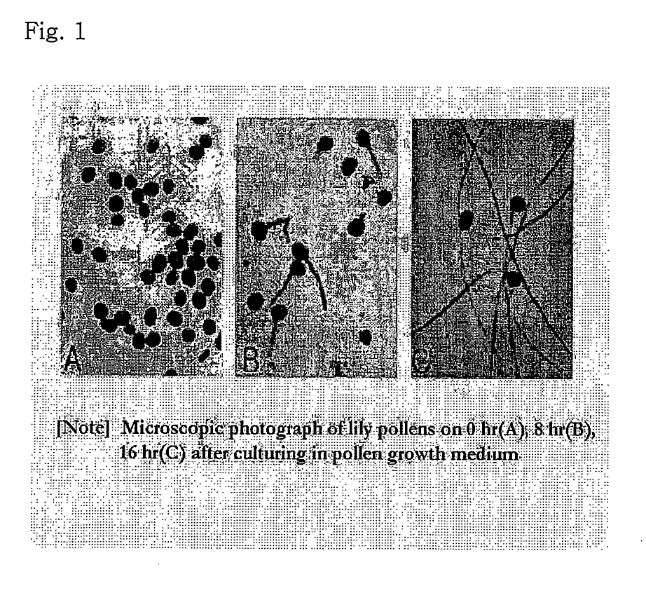

FIG. 1 is a microscopic photograph showing elongation of lily pollen tubes

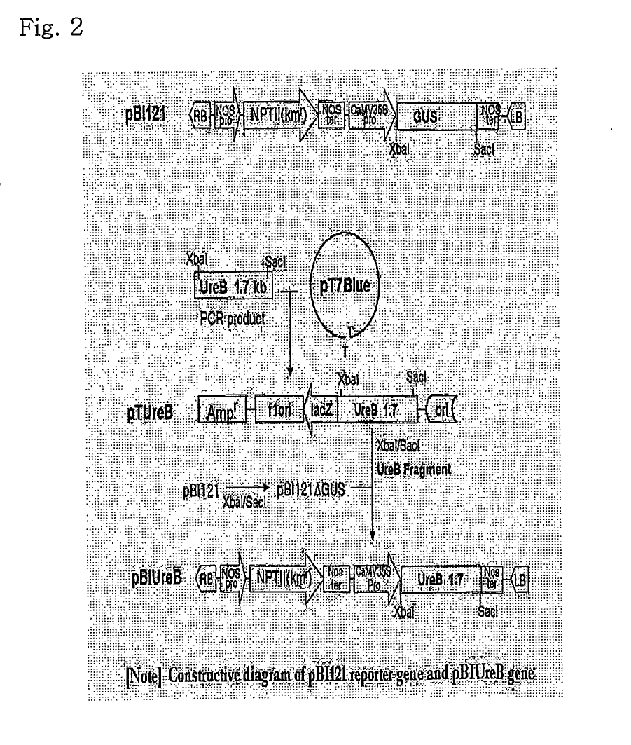

FIG. 2 shows maps of pBIUreB with ureB and pBI121 with GUS reporter gene

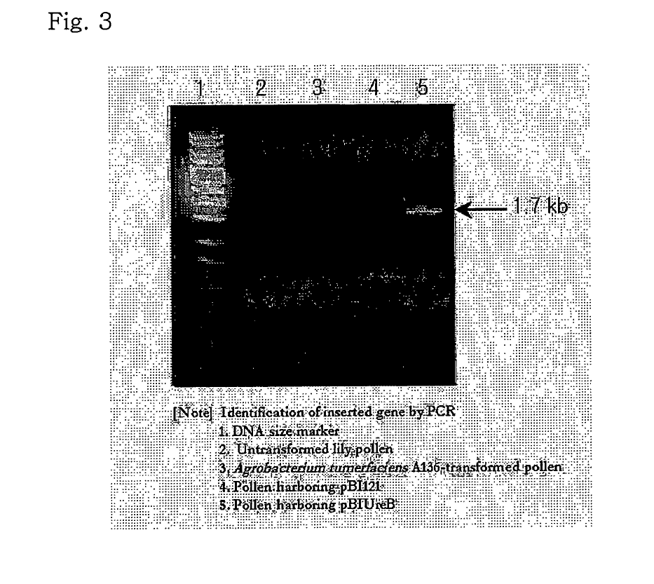

FIG. 3 shows the result of identifying ureB gene in pollen using the PCR technique

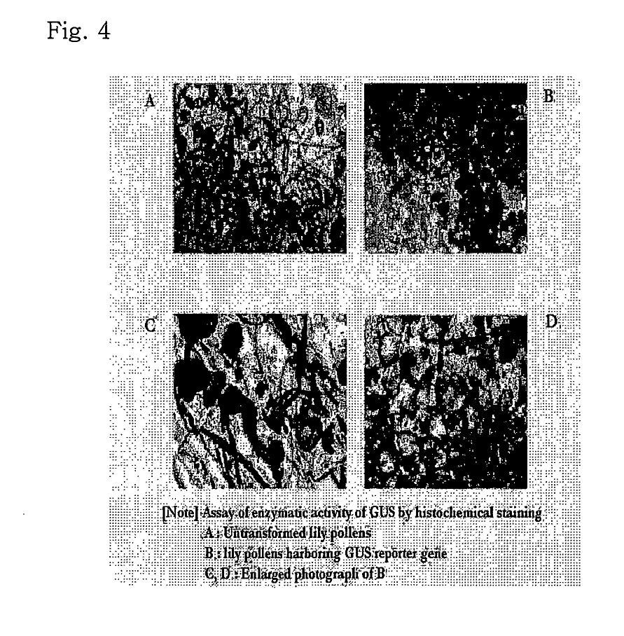

FIG. 4 is a microscopic photograph showing expression of GUS enzyme in pollen

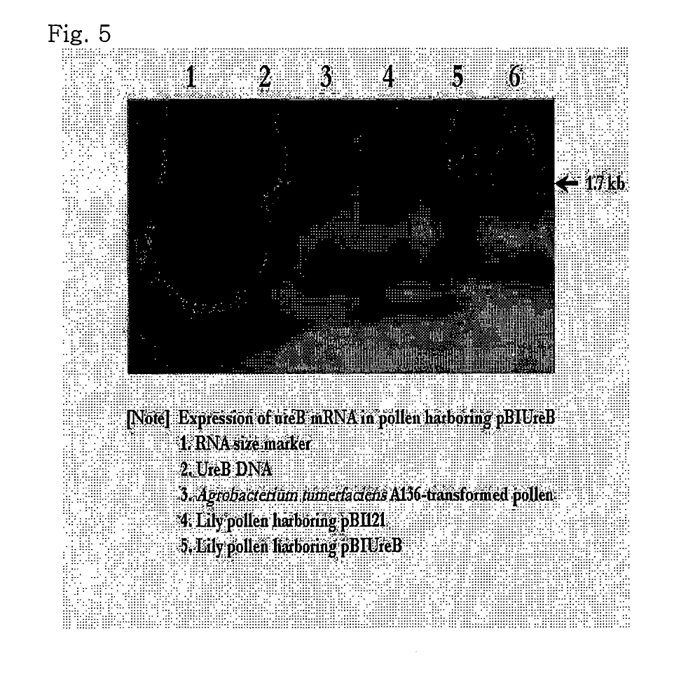

FIG. 5 is a photograph showing expression of ureB mRNA in pollen

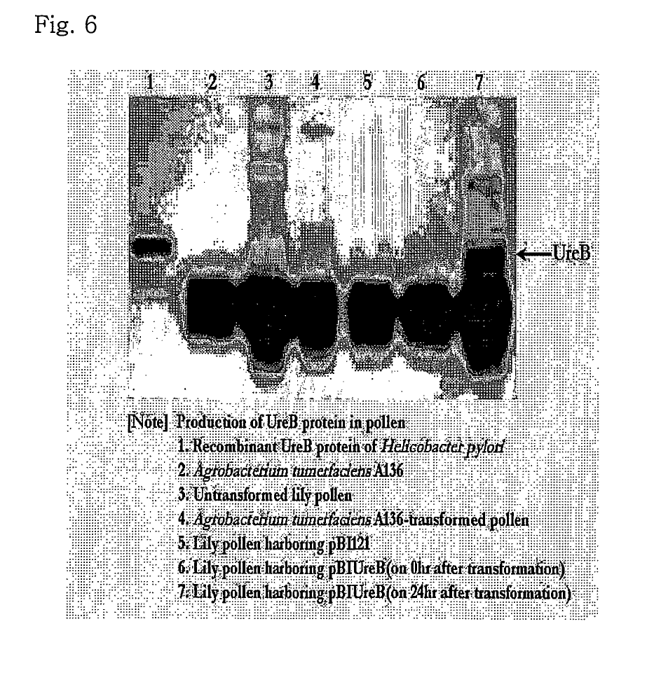

FIG. 6 shows expression of UreB protein in pollen

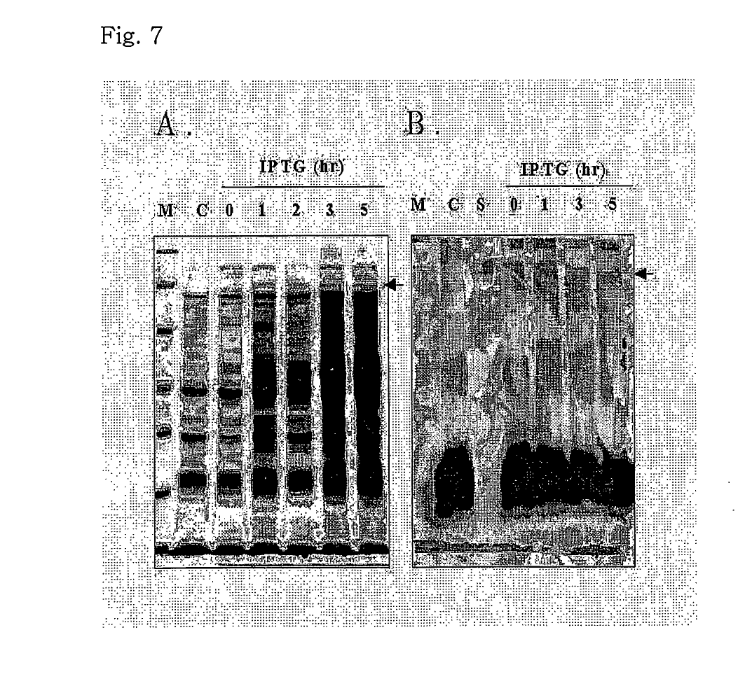

FIG. 7 is a photograph showing expression of tPA cDNA in E. coli treated with IPTG by SDS-PAGE

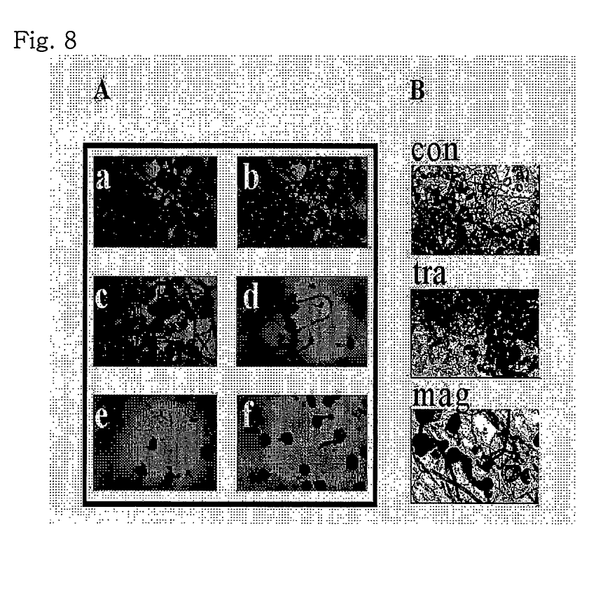

FIG. 8 shows kanamycin toxicity to growth of lily pollen and the result of histochemical staining

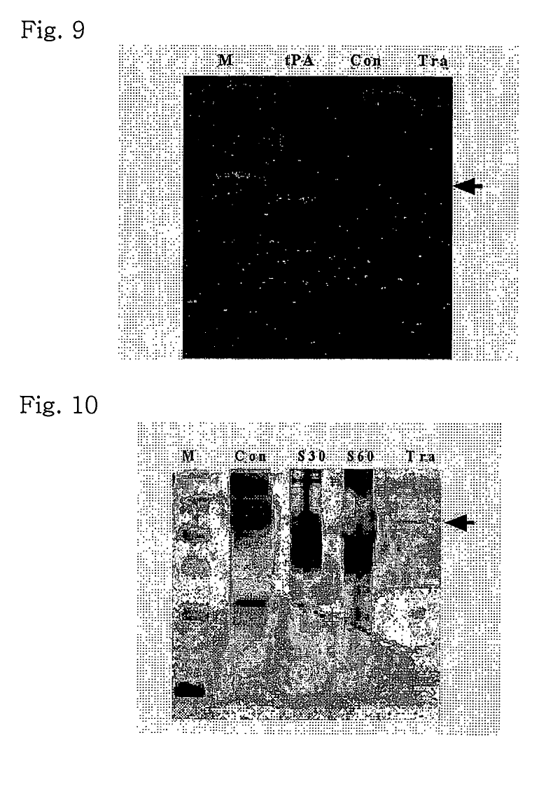

FIG. 9 shows a result of Southern hybridization with genomic DNA of pollen

FIG. 10 shows a result of Western blotting of pollen protein

### BEST MODE FOR CARRYING OUT THE INVENTION

### EXAMPLE 1

### Expression of ureB Gene Using Lily Pollen

To obtain the pollen for the present invention, dehisced anthers were detached from lily flowers (Lilium longiflorum) and their pollen grains were collected and then stored in a deep freezer at −70° C. until use. 1 g of the pollen grains were suspended in 200 mL of PGM composed of 7% sucrose, 1.27 mM Ca(NO₃)₂, 162 μM H₃BO₃, 0.99 mM KNO₃ and 3 mM KH₂PO₄ and incubated at 27° C. for 3 hours in the dark. The result of the elongation of lily pollen according to times in growth medium is represented in FIG. 1.

To observe the expression of the gene in pollen, two kinds of recombinant plasmid were prepared. One of them was pBI121 with GUS reporter gene (Clontech) and the other was pBIUreB harboring ureB gene from Helicobacter pylori (Genbank accession number AF352376: SEQ. No. 3). It has also been reported that recombinant UreB protein has an efficacy as anti-cancer vaccine. Processes for constructing pBIUreB are as following:

Primer No. 1: 5′-ATC CTA GAA TGA AAA AGA TTA GCA-3′(SEQ. No. 1) and primer No. 2: 5′-GAG CTC CTA GAA AAT GCT AAA GAG-3′(SEQ. No. 2) were synthesized. From pH808 harboring urease gene from Helicobacter pylori (Lee et al., J. Biochem. Mol. 31, 240, 1998), 1.7 kb ureB DNA fragment (SEQ. No. 5) was amplified by PCR. The ureB DNA fragment was fused to pT7 Blue T-vector (Novagene) to construct pTUreB. The resulting 1.7 kb ureB DNA obtained from pTUreB was excised by cutting with Xba I and Sac I was inserted to GUS-DNA deleted pBI121 by cutting with Xba I and Sac I to obtain pBIUreB. The recombinant diagram was represented in FIG. 2.

To introduce the target gene via Agrobacterium transformation and vacuum infiltration, the plasmids pBI121 and pBIUreB were transformed into Agrobacterium tumefaciens A136(ATCC 51350) using the freeze-thaw method, respectively. Following this, they were selected and incubated in LB media with kanamycin.

To introduce the gene, the transformed Agrobacterium tumefaciens harboring pBI121 and pBIUreB, respectively, were mixed with pollen pre-incubated for 3 hours, and then vacuum infiltration was carried out. The transformed Agrobacterium was grown in LB liquid medium with 50 mg/L of kanamycin and streptomycin, respectively. The bacterial cells were collected by centrifuging 1 mL of the culture solution at 10,000×g for 3 min. and suspended in the pollen growth medium, including 50 mg/mL of pollen. The suspended solution was placed in a vacuum container under vacuum condition (−80 Pa, for 20 min) and filtered by filter paper. The pollens were washed twice with PGM with 200 mg/L of cefotaxime. The collected pollen were grown in 10 mL of PGM at 27° C. for 20 hours.

### EXPERIMENTAL EXAMPLE 1

### Identifying Target Gene

To identify the target gene, fully-grown pollen tubes were collected. Genomic DNA was extracted from the pollen and PCR was performed for 35 cycles of the following reaction using the genomic DNA as a template; 94° C. 40 sec, 65° C. 1 min. 30 sec, 72° C. 2 min. Primers of SEQ. No. 1 and SEQ. No. 2 were used to identify ureB DNA. The resulting DNA was identified in 1% agarose gel electrolysis.

As shown in FIG. 3, the 1.7 kb DNA fragment from the genomic DNA of the transformed lily pollen was amplified and is not shown in the untransformed pollen and in pBI121 transformed pollen.

### EXPERIMENTAL EXAMPLE 2

### Assay of Enzymatic Activity of GUS in Pollen

To confirm the expression of the target gene from the transformed pollen via vacuum infiltration, the enzymatic activity of β-glucronidase expressed from pBI121 was measured by histochemical staining. In more detail, the fully-grown pollen were suspended in X-gluc solution(100 μL of 100 mM NaPO₄, 50 μL of X-gluc(1 mg/mL of stock solution) and 850 μL of sterile water), placed for 3 hours in the dark until a blue color came into view and then observed under microscope. The results were represented in FIG. 4.

As shown in FIG. 4, the untransformed pollen showed pale blue and the transformed pollen harboring pBI121 showed dark blue, which can confirm the expression of GUS gene.

### EXPERIMENTAL EXAMPLE 3

### Expression of UreB Gene in Pollen by Northern Blotting

To monitor the expression of ureB gene in the transformed pollen harboring pBIUreB, the production of ureB mRNA was investigated using Northern blotting. PTUreB was digested with Xba I and Sac I and the resulting 1.7 kb DNA fragment was used as a probe. Probe labeling was carried out according to the manual of the random primed DNA labeling kit (Reche Molecular Biochemicals). RNA of fully-grown pollen tubes was extracted in extraction buffer with guanidine isothiocyanate and was electrophoresced on 1% formaldehyde gel. The gel was transferred to a Nytran membrane (Schleicher & Schuell) and blotted with the probe. Hybridization was performed at 42° C. for 24 hours in a buffer containing 5×SSC, 0.1% N-laurylsarcosin, 0.02% SDS, 2% blocking agent and 30% formamide, and washed with buffer containing 0.5×SSC and 0.5% SDS. After this, the results were monitored by X-ray autoradiography. The results are represented in FIG. 5.

As shown in FIG. 5, 1.7 kb ureB mRNA was not expressed in the untransformed pollen, in pollen transformed by Agrobacterium and in pollen harboring pBI121, but was expressed in pollen harboring pBIUreB.

### EXPERIMENTAL EXAMPLE 4

### Expression of UreB Gene in Pollen by Western Blotting

Pollen samples were ground using a mini-pestle and suspended in an extraction buffer (KPO₄ pH 7.6, 2 mM PMSF and 10 mM EDTA). The pollen lysates were centrifuged at 13,000×g for 10 min and the supernatant was collected. According to Lowry's method, total protein was measured quantitatively. 30 μg of protein was used for Western blotting assay. The extracted protein was electrophoresced on SDS-8% polyacrylamide gel and the gel was transferred to Hybond-P-membrane (Amersham) in a transfer solution (0.025 M Tris-HCl, pH 8.3, 0.15 M glycine and 20% SDS) for 3˜4 hours at 300 mA followed with Western blotting. The primary antibody used a rabbit anti-H. pylori urease IgG and the second antibody used a goat anti-rabbit IgG HRP-conjugate. Results were monitored by the ECL detection system (Amersham) and represented in FIG. 6. 67 kDa UreB protein was used as a control of recombinant ureases from Helicobacter pylori.

As shown in FIG. 6, a band in the same position as that of 67 kDa UreB protein of the control was not shown in untransformed pollen, in pollen infected by Agrobacterium and in pollen harboring pBI121, whereas UreB protein was expressed successfully in pollen harboring pBIUreB. Meanwhile, 0.05% of UreB protein of the total soluble protein could be estimated through densitometric analysis in comparison to Western blotting using the recombinant UreB protein from bacteria. The yield of UreB protein was calculated in 50 μg UreB protein per 1 g of pollen.

### EXAMPLE 2

### Expression of tPA Gene Using Plant Pollen

rotein encoded by the [symbol] employing the lily pollen according to the present invention, tissue plasminogen activator (tPA) from human was expressed in transformed plant pollen.

According to the same method described in Example 1, lily pollen grains were collected and suspended in PGM composed of 7% sucrose, 1.8 mM Ca(NO₃)₂ and 1.6 mM H₃BO₃, and incubated at 27° C. for 16 hours in the dark.

For transformation, recombinant pBI/tPA and pSK/tPA constructs were constructed through the combination of pBI121 with tPA from human. The process for making the constructs are as follows: the primer No. 3 (sense) was designed to have Xba I restriction site fused to the start codon of tPA nucleotide sequence, 5′-AATCTAGACATGGATGCAATGAAGA-3′ and the primer No. 4 (antisense) was designed to contain the stop codon followed by SacI site, 5′-ATGATCTCTGGTCACGGTCGCATGTT-3′; From pETPFR(ATCC #40403) harboring tPA gene, tPA was amplified using tPA specific primers by PCR techniques. Polymerase Chain Reaction (PCR) was performed for 30 cycles of the following reaction: 94° C. 30 sec, 53° C. 30 sec, 72° C. 2 min. The resulting 1.7 kb DNA fragment (SEQ. No. 6) was ligated to pT7BlueR (Novagen) plasmid to obtain the recombinant pT/tPA. From it, the tPA DNA was excised by cutting with XbaI and SacI to be fused to pBluescript II SK (Stratagene) and to GUS-DNA deleted pBI121 (Clonetech) by digesting with XbaI and SacI to obtain pSK/tPA and pBI/tPA (not shown). After this, two kinds of recombinant plasmid were introduced into E. coli DH5α and E. coli XL-1 Blue. And then, it was identified that the sequence inserted in the resulted 1.7 kb DNA fragment was tPA full length sequence.

Introduction of the recombinant plasmid pBI121 and pBI/tPA or pSK/tPA constructs into Agrobacterium and induction of target gene via vacuum infiltration were performed according to the method described in Example 1. Also, Agrobacterium tumefaciens LBA4404 transformed with the recombinant plasmid were grown in LB medium with kanamycin and cefotaxime. Concentration of kanamycin and cefotaxime and incubation condition of the Agrobacterium tumefaciens LBA4404 transformed was carried out in the same manner described in Example 1.

### EXPERIMENTAL EXAMPLE 1

### Expression of tPA in E. coli

To confirm whether the 1.7 kb DNA fragment from the recombinant plasmid constructed in Example 2 encodes tPA full length, E coli XL-1BlueR cells transformed with the pSK/tPA were grown to the early log phase (OD₆₀₀=0.5) in LB medium in shaker flasks and then treated with IPTG to a final concentration of 2 mM for further cultivation. The bacterial cells were collected by centrifugation at 12,000×g for 5 min at 4° C. and suspended in extraction buffer (50 mM Tris-Cl, pH 7.5, 5 mM EDTA, 0.1% Tween 80, 2 mM PMSF) for disruption for three times in 2 min of sonication on ice. From the cell lysates, the inclusion body was collected by removing the supernatant following centrifugation at 12,000×g for 20 min at 4° C., and then used for SDS-PAGE and immuno-blotting. The LacZ-fused tPA was calculated to be a 66 kDa protein (30 amino acids from the LacZ-α plus 562 amino acids of 62,900 Da tPA protein), approximately. Human tPA standard was paralleled to show its slightly larger size than the induced protein band. FIG. 7A represents a result of SDS-PAGE of inclusion bodies from IPTG-treated E. coli harboring pSK/tPA for 0˜5 hrs, wherein M represents protein size marker, C represents expression of tPA cDNA from pBluescript SK II transformed. FIG. 7B represents a result of Western blotting of the membrane-transferred bacterial proteins.

As shown in FIG. 7, although not strong, the induced synthesis of the 66 kDa protein evidently appeared at the one-hour point of the IPTG treatment and continued to several hours examined in this experiment. From this result, the induced protein was almost expected to be the fused tPA, but its further confirmation was carried out by Western blotting.

### EXPERIMENTAL EXAMPLE 2

### Assay of Enzymatic Activity of GUS in Pollen by Histochemical Staining

In order to demonstrate the expression of target gene from the pollen, activity of GUS enzyme was assayed by histochemical staining according to experimental example 1 of Example 1. FIG. 8A represents kanamycin toxicity to elongation of pollen tubes, wherein a, b, c, d, e and f represent 0, 50, 100, 125, 150 and 200 mg/L of kanamycin added in the pollen growth media, respectively. FIG. 8B represents a result of histochemical staining of pollen tubes of the untransformed (con), pBI121 transformed (tra) and a magnified feature of the tra (mag).

As shown in FIG. 8, most of the transformed pollen grown with or without kanamycin was shown to be similarly demonstrating the GUS activity, implying that most of the pollen population successfully transformed by vacuum-infiltration.

### EXPERIMENTAL EXAMPLE 3

### Expression of tPA in Pollen by Southern Blotting

Fully-grown pollen tubes were washed several times with sterile distilled water, ground to a fine powder in the presence of liquid nitrogen using mortar and pestle, and used for extracting genomic DNA according to the CTAB/chloroform method (Taylor et al., 1993). Genomic DNA digested with XbaI and SacI was electrophoresced on a 0.8% agarose gel and transferred to a nylon membrane (Hybond N, Amersham) for hybridization with 1.7 kb tPA DNA. Probe labeling was done according to the ECL random prime system (Pharmacia). Hybridization was performed at 60° C. for 24 hrs in buffer containing 5×SSC, 0.1% SDS, 5% dextran sulfate, 100 μg/mL denatured sheared salmon sperm DNA and 5% Pharmacia liquid block. Results were monitored by ECL detection system (Pharmacia) following anti-fluorescein-HRP reaction.

As shown in FIG. 7, 1.7 kb DNA fragment was evident from the transformed, but none from the untransformed, strongly indicating tPA DNA integration into the pollen chromosomal DNA. Total soluble proteins estimated about 12% of the pollen weight were extracted for SDS-12% PAGE followed with Western blotting.

As shown in FIG. 9, protein bands in recognition to the tPA mAb could be observed at the location nearly identical to the standard tPA and expected to have a similar extent of glycosylation in mass in lily pollen. Approximately 0.05% of tPA protein of the total soluble proteins could be estimated through densitometric analysis in comparison to the standard tPA.

### EXPERIMENTAL EXAMPLE 4

### Expression of tPA in Pollen by Western Blotting

Expression of tPA recombinant protein was carried out by Western blotting. Pollen samples were ground in liquid nitrogen using a mortar and pestle and suspended in extraction buffer (50 mM Tris-Cl, pH 7.5, 5 mM EDTA, 0.1% Tween 80, 2 mM PMSF). The pollen lysates were centrifuged at 12,000×g for 15 min at 4° C. and the supernatant was used for SDS-12% polyacrylamide gel electrophoresis and Western blotting, according to the standard technique (Sambrook and Russell, 2001). The tPA protein was detected using mAb-tPA (Biodesign, ME) and Western blue stabilized substrate for alkaline phosphatase (Promega, WI). Single-chain tPA from human melanoma cell culture (Sigma, Mo) was used as a standard. FIG. 10 represents a result of Western blotting of pollen proteins, wherein M represents the protein size marker, Con represents pBI121-transformed pollen, S30 and S60 represent the standard tPA 30 ng and 60 ng, respectively and Tra represents pBI/tPA transformed.

As shown in FIG. 10, the transferred 66 kDa protein bands onto PVDF membrane could be detected as colored ones at their corresponding locations in recognition to mAb against human tPA. Taken these results together, the 1.7 kb PCR product amplified from pETPFR plasmid could be identified for its encoded tPA protein in size and immune reaction.

### INDUSTRIAL APPLICABILITY

As described before, the present invention comprises the use of plant pollen as a host for producing recombinant protein, which makes it possible to easily produce the recombinant protein at lower prices in comparison to production systems employing animal cell lines due to the absence of complicated costs in cell proliferation, maintenance of cellular genetic stability and operation of bioreactor.

Also, the present invention can directly produce recombinant protein in a pollen growth culture from a few hours to maximum of several days in a simple liquid medium supplemented with 3˜4 kinds of chemicals, including sugar. Therefore, the present invention is very useful in medical and genetic engineering industries.

1. A method for producing recombinant protein using plant pollen comprising the steps of:
isolating pollen from plant;
culturing the pollen in a pollen growth medium;
introducing a target gene into Agrobacterium vector to be transformed;
infecting the transformed Agrobacterium into the cultured plant pollen; and
growing the pollen with the gene in a pollen growth medium to obtain the resulting recombinant protein.

2. The method of production as set forth in claim 1, wherein the plant pollen is isolated from Lilium longiflorum or Pinaceae.

3. The method of production as set forth in claim 1, wherein the pollen growth medium is composed of 5˜10% sucrose, 0.5˜3 mM Ca(NO₃)₂, 50˜300 μM H₂BO₃, 0.001˜5 mM KNO₃ and 0.1˜10 mM KH₂PO₄.

4. The method of production as set forth in claim 1, wherein the gene is introduced into Agrobacterium by a method selected from the group consisting of particle bombardment, vacuum infiltration and electroporation.

5. The method of production as set forth in claim 1, wherein the target gene is UreB gene from Helicobacter pylori and tissue plasminogen activator (tPA) from humans.
